(19) United States
(12) Patent Application Publication
(10) Pub. No.: US 2018/0228832 A1
(43) Pub. Date: Aug. 16, 2018

(54) STOMATOLOGICAL COMPOSITION CONTAINING NSAID OR HEPARIN COMPOUND

(71) Applicant: Pharma Seeds Create, LLC, Hyogo (JP)

(72) Inventors: Toru HIBI, Hyogo (JP); Toru CHIBA, Nara (JP); Shigeru ENOMOTO, Hyogo (JP)

(73) Assignee: Pharma Seeds Create, LLC, Hyogo (JP)

(21) Appl. No.: 15/751,304

(22) PCT Filed: Aug. 18, 2015
(86) PCT No.: PCT/JP2015/073119

§ 371 Date: Feb. 8, 2018

(51) Int. Cl. A61K 31/727; A61K 31/5415; A61K 31/415; A61K 31/192; A61K 9/08; A61P 1/02; A61P 29/00

(52) U.S. Cl. CPC A61K 31/727; A61K 31/5415; A61K 31/415; A61P 29/00; A61K 9/08; A61P 1/02; A61K 31/192

(57) ABSTRACT

The present invention relates to the development of a stomatological composition and a kit each comprising an NSAID such as ibuprofen, a heparin compound, or a pharmaceutically acceptable salt thereof for the prevention, alleviation, or treatment of inflammations associated with oral cavity diseases and/or pains/infections associated with the inflammations and of inflammatory pain diseases/infections of the pharynx and/or the esophagus, and to a method for producing the composition and a method of use of the composition for the production of a medicament.

### TECHNICAL FIELD

The present invention relates to a stomatological composition and a kit each comprising an NSAID, a heparin compound, or a pharmaceutically acceptable salt thereof for the prevention, alleviation, or treatment of inflammations associated with oral cavity diseases and/or pains/infections associated with the inflammations and of inflammatory pain diseases/infections of the pharynx and/or the esophagus. The present invention also relates to a method for producing the composition and a method of use of the composition for the production of a medicament.

### BACKGROUND ART

Ibuprofen is a propionic acid-based acidic compound found in the 1960s by Boots in UK (current Abbott Laboratories) and is one of non-steroidal anti-inflammatory drugs (hereinafter called NSAIDs). NSAIDs are classified into several groups, and the propionic acid compound group containing ibuprofen includes loxoprofen known as Loxonin (trade name) and flurbiprofen used in plasters and the like. NSAIDs further include a phenylic acid group including diclofenac and indomethacin, a salicylic acid group including aspirin, an oxicam group including piroxicam and meloxicam, and a coxib group including celecoxib and rofecoxib. Various NSAIDs and pharmaceutically acceptable salts thereof exhibit anti-inflammatory analgesic activity and thus are widely used as pharmaceutical products.

Heparin is an acid mucopolysaccharide widely found in tissues of higher animals, especially, in muscles, the liver, the lung, the spleen, blood, and the like, has a lot of sulfuric acid groups in the molecule thereof, and thus interacts with various physiologically active substances. For this reason, in addition to heparin, for example, a heparinoid, which is a mucopolysaccharide polysulfate prepared by sulfating a polysaccharide having a repeating unit of a disaccharide composed of D-glucuronic acid and N-acetyl-D-galactosamine, a low-molecular-weight heparin prepared by enzymatic or chemical treatment of unfractionated heparin, other heparin compounds, and pharmaceutically acceptable salts thereof are widely used in the fields of medical care, cosmetics and the like. Each substance is known and has anti-inflammatory activity.

Commonly, when an anti-inflammatory analgesic drug substance like an acid NSAID such as ibuprofen or a heparin compound as an acid mucopolysaccharide is held in the mouth, strong irritating bitterness may be sensed, and thus various methods for reducing the bitterness has been developed. For example, commercial soft capsules containing an ibuprofen solution are prepared by encapsulation so as not to leak the content fluid in the mouth at the time of administration. Commercial ibuprofen tablets are coated tablets prepared by coating the tablet surface with a sugar or a polymer film. In each case, the active ingredient NSAID compound itself has not been subjected to treatment for reducing the bitterness. Hence, when the content of a soft capsule or a sugar-coated tablet is taken out of the capsule and held in the mouth for sensory testing (experimental example described later), irritating, stabbing pain and bitterness are sensed.

These ibuprofen preparations such as soft capsules and coated tablets are produced on the assumption that they are orally administered and ibuprofen as the active ingredient is absorbed from the gastrointestine and taken into the body. After a therapeutic dose of ibuprofen enters the systemic blood circulation and an intended blood concentration is achieved, ibuprofen exerts a pharmaceutical effect in accordance with its pharmacokinetic behavior.

In an embodiment of the method of use in the present invention (disclosed later), a preparation is held in the mouth while swished around for contact with the whole or part of the mouth and, after a while, spat out (in the specification, this procedure is sometimes called “oral rinsing” or “rinsing”), but such a method of use is not based on the assumption that ibuprofen as the active ingredient is orally administered and then taken into the body, and is different from that intended for conventional ibuprofen preparations.

The prior art relating to administration of non-steroidal anti-inflammatory drugs (NSAIDs) for oral cavity diseases such as stomatitis includes the treatment of intractable stomatitis by an indomethacin spray and the prevention of stomatitis by an azulene or Voltaren gargle water (Non-Patent Literature 1), the treatment of oral mucositis induced by cancer therapy (Non-Patent Literature 2); an example using ice balls (Non-Patent Literature 3); and a diclofenac gargle having an anti-inflammatory analgesic effect on post-operative pain (Non-Patent Literature 4). However, it is a well-known fact in the field that a patient orally administered with ibuprofen can suffer from stomatitis as a side effect (Non-Patent Literature 5).

As described above, there are previous reports on the administration of some NSAIDs including indomethacin and diclofenac for stomatitis and oral cavity diseases. However, there is no report or the like showing the effectiveness of buccal/oral cavity administration of ibuprofen, which is an NSAID most generally used in the world and considered to be highly safe, against stomatitis and other oral cavity diseases, oral injuries, and post-operative pain.

This is naturally understood from common technical knowledge that a patient orally administered with ibuprofen can suffer from stomatitis as a side effect. In addition, ibuprofen is safer than indomethacin and diclofenac but has lower efficacy, and thus a larger amount of ibuprofen is required to be administered. Moreover, ibuprofen tastes bad, and for these reasons, it is naturally understood that no buccal/oral cavity administration has been reported.

### CITATION LIST

### Non-Patent Literature

Non-Patent Literature 1: Byouin Yakkyoku Seizai Jireishu (Summary of Hospital Preparations), under supervision of Japanese Society of Hospital Pharmacists, Yakuji Nippo Ltd.
Non-Patent Literature 2: Kenji Momo, Yakuzaigaku (Journal of Pharmaceutical Science and Technology, Japan), 72 (1) 15-19, 2012.
Non-Patent Literature 3: Suzuki, Prevention of stomatitis by chemical treatment, Nihon Kangogakkai Ronbunshu 2, Seijin Kango, 34, 21-23, 2003, Japanese Nursing Association Publishing Company
Non-Patent Literature 4: Agarwal S. et al., Indian J. Dent. Res. 2010, Jul.-Sep. 21 (3); 408-412
Non-Patent Literature 5: Interview form of Brufen, p. 22, Section under “Safety” (3) Other side effects (Kaken Pharmaceutical Co., Ltd., revised May 2012 (Seventh edition))

### SUMMARY OF INVENTION

### Technical Problem

The present invention is intended to develop a stomatological composition and a kit each comprising an NSAID including ibuprofen, a heparin compound, or a pharmaceutically acceptable salt thereof for the prevention, alleviation, or treatment of inflammations associated with oral cavity diseases and/or pains/infections associated with the inflammations and of inflammatory pain diseases/infections of the pharynx and/or the esophagus. The invention is also intended to find a method for producing the composition and a method of use of the composition for the production of a medicament.

### Solution to Problem

There is common technical knowledge that the oral administration of ibuprofen causes stomatitis, whereas ibuprofen is an NSAID that is most generally used in the world and is considered to be highly safe. The inventors of the present invention have repeatedly performed intensive studies on whether ibuprofen can be directly administered buccally or to oral cavity for the prevention, alleviation, or treatment of oral cavity diseases. As a result, the inventors have found a stomatological composition that comprises ibuprofen and a pharmaceutically acceptable carrier and is for the prevention, alleviation, or treatment of inflammations associated with oral cavity diseases and/or pains associated with the inflammations.

The inventors have further found that not only ibuprofen but also meloxicam, loxoprofen, celecoxib, and flurbiprofen known as NSAIDs and heparin compounds known as an anti-inflammatory agent have similar advantageous effects, and have completed the present invention.

In other words, the present invention relates to the following aspects.

(1) A stomatological composition for prevention, alleviation, or treatment of an inflammation, a pain, or an infection associated with an oral cavity disease and/or an inflammatory pain disease or an infection of an upper respiratory tract, a pharynx, and/or an esophagus, the composition comprising an NSAID, a heparin compound, or a pharmaceutically acceptable salt thereof, and at least one pharmaceutically acceptable carrier.
(2) The composition according to the above (1), wherein the NSAID is at least one selected from the group consisting of ibuprofen, meloxicam, loxoprofen, celecoxib, and flurbiprofen, or the heparin compound is at least one selected from the group consisting of heparin, a heparinoid, and a low-molecular-weight heparin.
(3) The composition according to the above (1) or (2), wherein the composition is in the form of solution, gel, gel, patch, dentifrice, jelly, spray, gum, suspension, semisolid preparation, orally disintegrating tablet, chewable tablet, granule, effervescent tablet, or troche.
(4) The composition according to any one of the above (1) to (3), wherein the oral cavity disease is at least one selected from the group consisting of an oral injury, stomatitis, a periodontal disease, a tooth pain, a pain after tooth extraction, a post-operative pain, gingivitis, tooth hypersensitivity, an inflammatory disease, an inflammatory tongue pain, an infection (in an oral cavity or of a pharynx or an esophagus), an inflammation and a pain associated with dry mouth, and an inflammatory pain of a pharynx and/or an esophagus.
(5) The composition according to any one of the above (1) to (4), further comprising at least one additive selected from the group consisting of water, a solvent, a gelling agent, a pH adjuster, a solubilizer, a moisturizer, a thickener, a suspending agent, a flavoring agent, a sweetening agent, a corrigent, a coloring agent, an antiseptic agent, a surfactant, an emulsifier, and a stabilizing agent.
(6) The composition according to any one of the above (1) to (5), further comprising an additional pharmacologically active ingredient.
(7) The composition according to any one of the above (1) to (6), wherein the composition is a gargle.
(8) The composition according to any one of the above (2) to (7), wherein 100 g of the composition contains 100 μg to 20 g of ibuprofen, 2.5 μg to 500 mg of meloxicam, 30 μg to 6 g of loxoprofen, 67 μg to 13.3 g of celecoxib, or 20 μg to 4 g of flurbiprofen.
(9) The composition according to any one of the above (2) to (7), wherein 100 g of the composition contains 0.2 to 40,000 units of heparin, 0.2 to 40,000 international units of the low-molecular-weight heparin, or 1.5 μg to 3 g of the heparinoid.
(10) The composition according to any one of the above (1) to (9), wherein irritation and/or bitterness is reduced.
(11) A method for producing the composition according to any one of the above (1) to (10), the method comprising mixing an NSAID, a heparin compound, or a pharmaceutically acceptable salt thereof with at least one acceptable carrier.
(12) Use of the composition according to any one of the above (1) to (10) for the production of a medicament.
(13) A kit used for prevention, alleviation, or treatment of an inflammation, a pain, or an infection associated with an oral cavity disease and/or an inflammatory pain disease or an infection of an upper respiratory tract, a pharynx, and/or an esophagus, the kit comprising the composition according to the above (1).

In consideration of common technical knowledge that NSAIDs such as ibuprofen cause stomatitis, it is surprising that the stomatological composition of the present invention exerts marked effects on inflammations associated with oral cavity diseases including stomatitis and/or pains associated with the inflammations.

As used herein, the term “NSAIDs” may be acid NSAIDs such as ibuprofen, meloxicam, loxoprofen sodium, celecoxib, and flurbiprofen among non-steroidal anti-inflammatory drugs, may include basic NSAIDs such as tiaramide hydrochloride and tinoridine hydrochloride, or may be a combination of them.

As used herein, the term “heparin compound” includes heparin (including salts such as a sodium salt), a heparinoid, and a low-molecular-weight heparin. Here, the heparin typically has a molecular weight of about 5,000 to 30,000, whereas the low-molecular-weight heparin has a molecular weight of about 4,000 to 8,000, but the heparin compound is not limited to them.

As used herein, the term “pharmaceutically acceptable salt” or simply “salt” includes inorganic salts selected from salts with sodium, potassium, lithium, calcium, magnesium, aluminum, and the like; and organic salts such as salts with lidocaine, meglumine, trometamol, ornithine, arginine, lysine, diethanolamine, triethanolamine, and the like. Such a salt includes stereoisomers, optical isomers, and/or mixtures of these isomers, solvates, amorphous forms, polymorphic forms, and isotope-labeled forms as long as these are pharmaceutically acceptable. The present invention includes, within its scope, all possible stoichiometric and non-stoichiometric salts.

As used herein, the term “pharmaceutically acceptable carrier” is well established in the pharmaceutical field, therefore the conventional definition can be applied also in the invention. Such a pharmaceutically acceptable carrier includes, for example, water, ethanol, glycerol, polyethylene glycol, propylene glycol, methyl cellulose, hydroxypropyl methyl cellulose, hydroxypropyl cellulose, polyacrylic acid, polyvinylpyrrolidone, polysaccharide gum-based natural polymers, and appropriate oils and waxes such as vaseline, squalane, paraffin, and beeswax, but is not limited to them.

As used herein, the term “oral cavity disease” includes oral injuries, periodontal diseases, gingivitis, tooth hypersensitivity, oral inflammatory diseases including inflammations caused by contact of artificial teeth or orthodontic appliances, inflammatory tongue pains, other inflammatory conditions of oral tissues represented by stomatitis, decayed teeth, necrotizing ulcerative gingivitis, pains and/or inflammations associated with such diseases, halitosis associated with such diseases, viral diseases including herpetic lesions, inflammations that may develop after dental procedures such as bone surgery, tooth extraction, periodontal flap surgery, dental implantation, and scaling and root planing and/or pains and infections associated with the inflammations, and inflammatory pains and/or infections of the pharynx/esophagus. In particular, the oral cavity disease also includes dentoalveolar infections, dental abscesses (for example, cellulitis of the jaw; osteomyelitis of the jaw), acute necrotizing ulcerative gingivitis, that is, Vincent's infection, infectious stomatitis, that is, acute inflammation of the buccal mucosa, and noma, that is, gangrenous stomatitis or cancrum oris. Injuries in the oral cavity and pains and/or inflammations after oral surgery are further included. Oral and dental infections mean infections caused by bacteria, fungi, viruses, or the like, as specifically disclosed in Finegold, Anaerobic Bacteria in Human Diseases (chapter 4, pp 78-104, and chapter 6, pp 115-154, Academic Press, Inc., 1977). The stomatological composition of the present invention is particularly effective on the treatment and prevention of oral injuries, stomatitis, periodontal diseases, tooth pains, pains after tooth extraction, hyperesthesia, or pains and/or inflammations after oral surgery. In particular, it should be noted that the stomatological composition of the present invention has marked effects on, for example, oral cavity diseases induced by the administration of a medicament such as an anticancer agent and diseases of the pharynx/esophagus.

As used herein, the term “stomatological composition” preferably means a product of a particular therapeutic agent that is usually not only used for the purpose of systemic administration but also held in the oral cavity over enough time for substantial contact with the whole or part of the tooth surface and/or the oral cavity tissue for the purpose of exerting the activity in the oral cavity. For example, after held in the oral cavity, the composition may or may not be swallowed.

The stomatological composition of the present invention may be called gargle (oral rinse), mouth rinse, collutory, mouthwash, oral care product, preparation for the oral cavity, dental liquid, or other names commonly used in the field.

The stomatological composition of the present invention can be in various forms of liquid such as aqueous solution, gel, patch, dentifrice, jelly, spray, mousse, foam, gum, suspension, semisolid preparations, orally disintegrating tablets, granules to be dissolved at the time of use, effervescent tablets, and chewable tablets as long as it can be used for rinsing followed by spitting out in an intended step. In particular, a solution form, a gel form, a spray form, or a patch form is preferred. The composition in any of these dosage forms may or may not be swallowed at the time of use. The semisolid preparation means ointments, creams, lotions, and liniments, for example. The composition may be in a troche form (in the case of troches, ibuprofen or flurbiprofen is excluded as the active pharmaceutical ingredient).

When the composition is a dentifrice, the dentifrice is typically used for hyperesthesia, periodontal diseases, gingivitis, and the like, but the application is not limited to them. The dentifrice may be what is called a toothpaste in a paste form or may be a tooth powder.

When the stomatological composition of the present invention is in a gel form, an additive such as a thickener can be used in order to give an intended viscosity. In addition, a moisturizer may be added in order to maintain the gel form. The gel can be prepared by a method known by a person skilled in the art. The stomatological composition in such a gel form can be used for rinsing followed by spitting out in an intended step as with other forms of stomatological compositions, and is included as an embodiment in the present invention. As for the thickener, for example, xanthan gum, carrageenan, hyaluronic acid, carboxymethyl cellulose, glycerol, sorbitol liquid, propylene glycol, polyethylene glycol, and the like can be used as the moisturizer. Other known additives can be appropriately used.

When being in a patch form, the stomatological composition of the present invention is preferably an adhesive patch that becomes gradually eroded, dissolved, and disintegrated by hydration of the molecules. Such a patch typically contains a binder and a base material for holding and releasing an NSAID. Examples of the binder or the base material include, but are not limited to, microcrystalline cellulose, isomalt, cornstarch, gelatin, carrageenan, xanthan gum, konjac gum, locust bean gum, guar gum, agar, gum arabic, cellulose derivatives such as hydroxypropyl methyl cellulose (HPMC), hydroxypropyl cellulose (HPC), and sodium carboxymethyl cellulose (CMC-Na), polyacrylic acid, polyvinyl alcohol, and pectin.

When the composition is in a patch form, any composition that becomes dissolved and disintegrated by hydration of the molecules and has adhesive properties is included in the scope of the invention. The composition of the present invention in such a patch form can be used for rinsing followed by spitting out in an intended step as with other forms of stomatological compositions, and is included as an embodiment in the present invention.

The stomatological composition of the present invention in the form of spray, mousse, foam, gum, suspension, semisolid preparations, tablets, orally disintegrating tablets, granules, chewable tablets, effervescent tablets, or the like can also be prepared by a method known by a person skilled in the art.

The stomatological composition in such a form can be used for rinsing followed by spitting out in an intended step, and is included as an embodiment in the present invention.

The composition of the present invention can contain a known additive commonly used in the field. Examples of the additive include, but are not limited to, water, a solvent, a gelling agent, a pH adjuster, a solubilizer, a moisturizer, a thickener, a suspending agent, a flavoring agent, a sweetening agent, a corrigent, a coloring agent, an antiseptic agent, a surfactant, an emulsifier, and a stabilizing agent. If desired, a fluoride ion source, an antilithic agent, an anticalculus agent, a polishing agent, a bleach, or the like may be added.

As other pharmacologically active ingredients usable in the present invention, for example, sedatives such as propylacetylurea and anhydrous caffeine, local anesthetics such as lidocaine, other anti-inflammatory agents such steroids, antimicrobial agents, antifungal agents, antiviral agents, mucosal tissue repairing agents, mucosa protecting agents, and salivating agents can be applied. Anti-inflammatory agents other than the NSAIDs, the heparin compounds, and the steroids can also be applied to the composition of the present invention. In addition, pharmaceutical products other than the above ingredients can be used in combination with and/or added to the composition of the present invention.

Examples of the water used for the preparation of the stomatological composition of the present invention include purified water, distilled water, and drinking water such as tap water, and a water having a low ion content with less impurities is preferred. In the composition of the present invention, the water content varies with the intended form of the composition, but can be about 1 to 99% by weight and preferably about 3 to 97% by weight relative to the total amount of the composition. The water content includes, for example, water contents held in other ingredients.

The gelling agent usable in the present invention may be any chemical substance capable of making a liquid into a gel, and, for example, high concentration micelle of a surfactant, cellulose polymers such as carboxymethyl cellulose, hydroxypropyl cellulose, and carboxymethyl hydroxyethyl cellulose or salts thereof, acrylic acid polymers such as polyacrylic acid or salts thereof, polyvinyl alcohols such as polyvinyl acetate, and natural polysaccharide gums such as locust bean gum and guar gum can be used. Glycol polymers such as macrogol (polyethylene glycol) and polypropylene glycol are also included, but these are non-limiting examples.

The content of the gelling agent is about 0.01 to 10% by weight, preferably about 0.1 to 5% by weight, and particularly preferably about 0.25 to 3% by weight relative to the total amount of the composition of the present invention.

When a gelling agent is used in the composition of the present invention, one or more thickeners can be added so that the composition has a preferred viscosity and stability or exerts an intended active ingredient-releasing property at the time of use. Examples of the thickener usable in the present invention include, but are not limited to, carboxyvinyl polymer, carrageenan, hydroxyethyl cellulose, LAPONITE, and cellulose compounds such as methyl cellulose, hydroxypropyl cellulose, hydroxypropyl methyl cellulose, carboxymethyl cellulose, and carboxymethyl hydroxyethyl cellulose or salts thereof. Natural gums such as karaya gum, xanthan gum, gum arabic, and tragacanth gum can also be used. In order to improve the texture, magnesium aluminum silicate or silica can also be used, for example.

The pH adjuster usable in the present invention may be any agent capable of adjusting the pH of the composition of the present invention to an intended value, and can be used in order to adjust the pH to a range of about 2.5 to 10.0, more preferably, about 5.0 to 8.5.

Preferred examples of the pH adjuster include alkali metal hydroxides, alkali metal oxides, carbonates, sesquicarbonates, borates, silicates, phosphates, organic acids, organic acid salts, and organic bases. Specific examples include anhydrous sodium monohydrogen phosphate, sodium dihydrogen phosphate, monosodium phosphate, trisodium phosphate, benzoic acid or salts thereof, salicylic acid or salts thereof, sodium hydroxide, potassium hydroxide, magnesium hydroxide, alkali metal carbonates such as sodium carbonate, potassium carbonate, and magnesium carbonate, alkali metal bicarbonates such as potassium hydrogen carbonate and sodium hydrogen carbonate (sodium bicarbonate), pyrophosphates, boric acid, citric acid, sodium citrate, succinates, maleates, fumarates, malic acid, tartaric acid and salts thereof, lactic acid, imidazole, triethanolamine, diethanolamine, diisopropanolamine, trometamol, meglumine, lidocaine, and salts thereof. Of them, more preferred are, for example, alkali metal hydroxides, alkali metal bicarbonates such as potassium hydrogen carbonate and sodium hydrogen carbonate, phosphates, organic acids, organic acid salts, glucosamine hydrochloride, trometamol, sodium lactate, sodium malate, sodium acetate, basic amino acids such as lysine, arginine, histidine, and tryptophan, and organic bases such as trometamol, meglumine, and lidocaine, but these are non-limiting examples.

The pH adjuster may be contained in an amount of about 0.01 to 50% by weight, preferably about 0.01 to 40% by weight, more preferably about 0.03 to 30% by weight, and even more preferably about 0.03 to 25% by weight relative to the total amount of the composition of the present invention.

As the solubilizer usable in the present invention, a solubilizer known in the field can be used. Examples include, but are not limited to, meglumine, sodium benzoate, nicotinic acid amide, ethylenediamine, and glycol compounds such as ethylene glycol and cellosolve.

The moisturizer usable in the present invention prevents the composition from being dried by exposure to air. The moisturizer may allow the composition to provide moisture feeling in the mouth, or some types of moisturizers may give the composition a desirable flavor. The moisturizer may be contained in an amount of about 0 to 70% by weight and preferably about 5 to 25% by weight relative to the total amount of the composition of the present specification. Preferred examples of the moisturizer include polyhydric alcohols such as glycerol, sorbitol, xylitol, butylene glycol, polyethylene glycol, and propylene glycol, and particularly preferred are sorbitol, glycerol, hyaluronic acid, and the like, but the moisturizer is not limited thereto.

Examples of the flavoring agent usable in the present invention include, but are not limited to, winter green oil, peppermint oil, spearmint oil, clove bud oil, menthol, anethole, methyl salicylate, eucalyptole, cassia, 1-menthyl acetate, sage, eugenol, parsley oil, oxanone, α-irisone, marjoram, lemon, orange, propenyl guaethol, cinnamon, vanillin, thymol, linalool, and cinnamaldehyde glycerol acetal (CGA). The flavoring agent is preferably used at a concentration of about 0.001 to 5% by weight relative to the total amount of the composition of the present invention.

Preferred examples of the sweetening agent usable in the present invention include, but are not limited to, sucrose, glucose, saccharin, glycyrrhizic acid, dextrose, fructose, lactose, mannitol, sorbitol, fructose, maltose, xylitol, honey, starch syrup, saccharin, thaumatin, aspartame, D-tryptophan, dihydrochalcone, acesulfame, and cyclamic acid and salts thereof, and particularly preferred are sodium cyclamate, saccharin sodium, and the like. The sweetening agent is used at a concentration of about 0.05 to 30% by weight and preferably about 0.1 to 10% by weight relative to the total amount of the composition of the present invention.

Examples of the corrigent usable in the present invention include, but are not limited to, ascorbic acid, citric acid, glycyrrhizic acid, glutamic acid, succinic acid, tartaric acid, fumaric acid, malic acid, taurine, sarcosine, glycyrrhizic acid and salts thereof, gambir, sweet hydrangea leaf, fennel, erythritol, sodium chloride, magnesium chloride, eugenol, Phellodendron bark extract, Coptis rhizome, orange oil, cacao, caramel, carbachol, Glycyrrhiza glabra, camphor, 5′-guanylic acid, chlorella extract, cinnamon extract, cinnamon oil, saffron, methyl salicylate, Zanthoxylum fruit tincture, peony extract, ginger, cinnamaldehyde, stevia extract, Swertia herb, sorbitol, cyclodextrin, soybean oil, jujube extract, taurine, tannic acid, clove oil, bitter orange peel extract, Picrasma wood extract, plum extract, honey, Mentha water, peppermint oil, menthol, povidone, borneol, malt extract, eucalyptus oil, green tea powder, lemon oil, rose oil, and royal jelly. The corrigent is used in an amount of about 0.05 to 30% by weight and preferably about 0.2 to 20% by weight relative to the total amount of the composition of the present invention.

Examples of the coloring agent usable in the present invention include, but are not limited to, coloring agents known to be safely used in the field of pharmaceutical products or foods, such as Blue No. 1, Yellow No. 4, Yellow No. 5, Red No. 2, Red No. 3, and Red No. 102. The coloring agent is preferably used in an amount of about 0.003 to 5% by weight relative to the total amount of the composition of the present invention.

Examples of the antiseptic agent usable in the present invention include, but are not limited to, benzoic acid and salts thereof, sodium edetate, agar, chlorhexidine salts, p-hydroxybenzoate esters (ester means methyl, ethyl, propyl, isopropyl, and butyl esters, for example), benzalkonium chloride, and benzethonium chloride. The antiseptic agent is preferably used in an amount of about 0.01 to 5% by weight relative to the total amount of the composition of the present invention.

The surfactant usable in the present invention is preferably a surfactant that is appropriately stable and foamable over a wide pH range (for example, about 2.5 to 10). The surfactant may be anionic, nonionic, amphoteric ionic, zwitterionic, or cationic.

Examples of the anionic surfactant preferably usable in the composition of the present invention include water-soluble salts of alkyl sulfates having 8 to 20 carbon atoms and water-soluble salts of sulfonatedmonoglycerides of fatty acids having 8 to 20 carbon atoms. Sodium alkyl sulfates, sodium lauryl sulfate, and sodium sulfonate are examples thereof. In addition, sarcosinates such as sodium lauroyl sarcosinate, taurates, sodium lauryl acetate, sodium lauroyl isethionate, sodium laureth carboxylate, sodium dodecylbenzenesulfonate, and mixtures thereof are preferably used, for example. U.S. Pat. No. 3,959,458 discloses many preferred anionic surfactants, which are also usable.

The nonionic surfactant preferably usable in the composition of the present invention can be broadly defined as compounds produced by the condensation of a hydrophilic alkylene oxide group with an aliphatic or aromatic compound. Preferred examples of the nonionic surfactant include, but are not limited to, poloxamer, polyoxyethylene alkyl ethers, polyoxyethylene sorbitan esters, polyoxyl hydrogenated castor oil, fatty alcohol ethoxylates, polyethylene oxide condensates of alkyl phenols, products prepared by the condensation of ethylene oxide with the reaction product of propylene oxide and ethylene diamine, ethylene oxide condensates of aliphatic alcohols, tertiary amine oxides, tertiary phosphine oxides, and dialkyl sulfoxides. Of them, poloxamer also functions as an emulsifier, a binder, and a stabilizer, reduces the astringency of metal ions, and thus is a particularly preferred surfactant. Poloxamers commercially available from BASF have a molecular weight ranging from about 1,000 to 15,000. Poloxamer 407 and Pluraflo L4370 are preferably used in the present invention.

The amphoteric surfactant preferably usable in the composition of the present invention includes, for example, aliphatic secondary or tertiary amine derivatives. The aliphatic residue may be linear or branched, has about 8 to about 18 carbon atoms, and contains an anionic water-soluble group such as a carboxylate, sulfonate, sulfate, phosphate, or phosphonate group. In addition, betaines, specifically, cocamidopropyl betaine can be used, and mixtures thereof can also be used. U.S. Pat. No. 4,051,234 discloses many nonionic and amphoteric surfactants, which are also usable in the composition of the present invention. The amphoteric surfactant is used in an amount of about 0.25 to 12% by weight, preferably about 0.5 to 8% by weight, and particularly preferably about 1 to 6% by weight relative to the total amount of the composition of the present invention.

Examples of the stabilizing agent usable in the present invention include, but are not limited to, adipic acid, ascorbic acid, sodium sulfite, sodium hydrogen sulfite, dibutylhydroxytoluene, butylated hydroxyanisole, sodium edetate, sodium chloride, citric acid, cyclodextrin, and cysteine.

As the emulsifier and the suspending agent usable in the present invention, the above surfactants can be preferably used. In addition, lecithins such as soybean lecithin, egg yolk lecithin, hydrogenated lecithin, and enzymatically decomposed lecithin, and higher alcohols such as cetanol, lauryl alcohol, stearyl alcohol, and lanolin alcohol are also usable.

As the antifungal agent usable in the present invention, for example, lower alcohols such as isopropyl alcohol and naturally derived ingredients such as chitosan, catechin, and polyphenols are preferably usable.

As the salivating agent usable in the present invention, for example, the ingredients exemplified above as the corrigent, cevimeline hydrochloride, pilocarpine hydrochloride, anethole trithione, alkaloids, other preparations known in the field, naturally derived ingredients such as lemon, pickled plum, and Chinese quince, and Kampo medicines such as byakko-ka-ninj in-to and bakumondo-to can be preferably used.

The stomatological composition of the present invention may further contain a biologically usable fluoride ion source. Examples of the fluoride ion source include, but are not limited to, sodium fluoride, tin fluoride, indium fluoride, amine fluorides, and sodium monofluorophosphate.

The composition of the present invention may contain about 50 to 3,500 ppm, preferably about 500 to 3,000 ppm of the fluoride ion source. In order to deliver an intended amount of fluoride ions, the fluoride ion source may be used in an amount of about 0.1 to 5% by weight, preferably about 0.2 to 1% by weight, and more preferably about 0.3 to 0.6% by weight relative to the total amount of the composition of the present invention.

In the present invention, the composition may contain titanium dioxide or a peroxide source as a dental bleach or an antimicrobial agent. The peroxide provides effects other than tooth whitening. Hydrogen peroxide, calcium peroxide, urea peroxide, and the like as the peroxide source are effective on therapeutic and/or preventive treatment against, for example, decayed tooth, dental plaque, gingivitis, periodontitis, halitosis, chronic recurrent aphtha, inflammations caused by artificial teeth, injuries by orthodontic appliances, traumatic oral lesions and mucosal infections after tooth extraction or after periodontal ligament surgery, and herpetic stomatitis.

The composition containing a peroxide exerts an action of generating oxygen bubbles by interaction with a tissue and salivary enzymes, and the swishing action of a mouth rinse enhances the oxygen-generating action. Such an action has an advantage to deriver other agents into gingival crevices, resulting in the prevention of colonization and multiplication of anaerobic bacteria known to be associated with periodontal diseases. Therefore, the peroxide is a preferred antimicrobial agent used in the present invention.

The peroxide source may be contained in an amount of about 0.01 to 10% by weight, preferably about 0.1 to 5% by weight, more preferably about 0.2 to 3% by weight, and most preferably about 0.3 to 0.8% by weight relative to the total amount of the composition of the present invention.

The composition of the present invention may contain an anticalculus agent. The anticalculus agent reduces the deposition of minerals associated with the formation of dental calculus, that is, what is called dental plaque deposition. As the anticalculus agent, a chelating agent having an action of decomposing dental plaque is preferred. Examples of the chelating agent include pyrophosphates, tripolyphosphates, and diphosphonates such as EHDP (ethane hydroxy diphosphonate) and AHP (azacycloheptane diphosphonate). Pyrophosphate salts useful as a pyrophosphate source include dialkali metal pyrophosphate salts and tetraalkali metal pyrophosphate salts. In addition, disodium dihydrogen pyrophosphate (Na₂H₂P₂O₇), tetrasodium pyrophosphate (Na₄P₂O₇), and tetrapotassium pyrophosphate (K₄P₂O₇) in anhydrous or hydrated forms are preferred. In the composition of the present invention, the pyrophosphate salt may be present in any of three forms: a predominately dissolved form, a predominately undissolved form, and a mixture of dissolved and undissolved forms.

Polymeric carboxylates are also used as a preferred anticalculus agent of the present invention. Examples include copolymers of maleic anhydride or maleic acid with a polymerizable ethylenic unsaturated monomer, such as methyl vinyl ether, methoxyethylene, styrene, isobutylene, and ethyl vinyl ether. These substances are well known in the field, and can be used in the form of, for example, a water-soluble alkali metal salt formed by neutralization of part, preferably the whole of the free acid moieties, including a potassium salt, preferably a sodium salt or an ammonium salt. For example, Gantrez series (methoxyethylene-maleic anhydride copolymers) of GAF Chemicals Corporation can be preferably used in the present invention.

The amount of the chelating agent preferably used in the present invention is about 0.1 to 2.5% by weight, preferably about 0.5 to 2.5% by weight, and more preferably about 1.0 to 2.5% by weight.

The polishing agent used in the composition of the present invention is preferably, for example, a commonly used dental substance that is miscible with other substances in the composition and does not excessively abrade the dentin.

Examples include, but are not limited to, silicas (such as silica gel and precipitated silica), insoluble sodium polymetaphosphate, hydrated alumina, calcium carbonate, dicalcium orthophosphate dihydrate, calcium pyrophosphate, tricalcium phosphate, calcium polymetaphosphate, hydroxyapatite, eggshells, and titanium oxide. Alternatively, a resin-based polishing agent such as a condensate of urea and formaldehyde can also be used. Examples of the resin herein include phenol resins, urea, crosslinked epoxides, and crosslinked polyesters.

As the silicas usable in the present invention, a silica having an average particle diameter of about 0.1 to 30 microns, preferably about 5 to 15 microns, can be typically used. Preferred examples include, but are not limited to, Syloid series of silica xerogel commercially available under the trade name Syloid from W. R. Grace & Company Davison Chemical Division and Zeodent series commercially available under the trade name Zeodent from J.M. Huber Corporation.

The total amount of the polishing agent in the composition of the present invention is typically about 6 to 70% by weight relative to the total amount of the composition, and the composition in a toothpaste form preferably contains about 10 to 50% by weight of the polishing agent. The composition of the present invention in a form other than a dentifrice typically preferably contains only a small amount of, for example, about 0.5 to 10% by weight of the polishing agent or contains no polishing agent.

The composition of the present invention is produced by appropriately selecting and mixing the above ingredients to form a preparation such as a gargle (oral rinse) as indicated in the present invention. As the above ingredients, for example, a commercial preparation of an NSAID such as ibuprofen or a commercial preparation containing a heparin compound may be used. When such a commercial preparation is used in the present invention, the additives and the like contained therein may be removed or the commercial preparation may be used as it is.

The amount of a compound contained in the composition of the present invention is not limited to particular values, but should be a safe and effective amount. The safe and effective amount used herein is an amount of an active ingredient that is safe enough for the tissue in the oral cavity and can sufficiently provide intended advantageous effects. The safe and effective amount of an NSAID as the active ingredient varies with the particular condition to be treated, the age and physical conditions of the patient to be treated, the severity of the condition, the duration of treatment, the nature of combination therapy, and the particular dosage form or carrier used. The amount used is appropriately adjusted depending on the purpose.

The composition of the present invention containing ibuprofen or a pharmacologically acceptable salt thereof typically contains ibuprofen or a pharmacologically acceptable salt thereof in an amount of about 100 μg to 20 g, preferably about 1 mg to 10 g, more preferably about 10 mg to 6 g, and even more preferably about 100 mg to 3 g relative to 100 g of the composition for adults. Such a composition can be used at once, in several portions, or repeatedly.

The composition of the present invention containing meloxicam or a pharmacologically acceptable salt thereof typically contains meloxicam or a pharmacologically acceptable salt thereof in an amount of about 2.5 μg to 500 mg, preferably about 25 μg to 250 mg, more preferably about 250 μg to 150 mg, and even more preferably about 2.5 to 75 mg relative to 100 g of the composition for adults. Such a composition can be used at once, in several portions, or repeatedly.

The composition of the present invention containing loxoprofen sodium (anhydride) or loxoprofen and a pharmacologically acceptable salt thereof typically contains loxoprofen sodium (anhydride) or loxoprofen in an amount of about 30 μg to 6 g, preferably about 300 μg to 3 g, more preferably about 3 mg to 1.8 g, and even more preferably about 30 to 900 mg relative to 100 g of the composition for adults. Such a composition can be used at once, in several portions, or repeatedly.

The composition of the present invention containing celecoxib or a pharmacologically acceptable salt thereof typically contains celecoxib or a pharmacologically acceptable salt thereof in an amount of about 67 μg to 13.3 g, preferably about 667 μg to 6.7 g, more preferably about 6.7 mg to 4 g, and even more preferably about 66.7 mg to 2 g relative to 100 g of the composition for adults. Such a composition can be used at once, in several portions, or repeatedly.

The composition of the present invention containing flurbiprofen or a pharmacologically acceptable salt thereof typically contains flurbiprofen or a pharmacologically acceptable salt thereof in an amount of about 20 μg to 4 g, preferably about 200 μg to 2 g, more preferably about 2 mg to 1.2 g, and even more preferably about 20 to 600 mg relative to 100 g of the composition for adults. Such a composition can be used at once, in several portions, or repeatedly.

The composition of the present invention containing a heparinoid or a pharmacologically acceptable salt thereof typically contains the heparinoid or a pharmacologically acceptable salt thereof in an amount of about 1.5 μg to 3 g, preferably about 15 μg to 1.5 g, more preferably about 150 μg to 900 mg, and even more preferably about 1.5 to 450 mg relative to 100 g of the composition for adults. Such a composition can be used at once, in several portions, or repeatedly.

The composition of the present invention containing a low-molecular-weight heparin or a pharmacologically acceptable salt thereof typically contains the low-molecular-weight heparin or a pharmacologically acceptable salt thereof in an amount of about 0.2 to 40,000 international units (IU), preferably about 2 to 20,000 IU, more preferably about 20 to 12,000 IU, and even more preferably about 200 to 6,000 IU relative to 100 g of the composition for adults. Such a composition can be used at once, in several portions, or repeatedly. The low-molecular-weight heparin may be a low-molecular-weight heparin known in the field.

The composition of the present invention containing heparin or a pharmacologically acceptable salt thereof typically contains heparin or a pharmacologically acceptable salt thereof in an amount of about 0.2 to 40,000 units, preferably about 2 to 20,000 units, more preferably about 20 to 12,000 units, and even more preferably about 200 to 6,000 units relative to 100 g of the composition for adults. Such a composition can be used at once, in several portions, or repeatedly.

Such a heparin compound may be a known one derived from pigs, cattle, or the like.

In the present invention, the composition of the present invention can be provided as a kit. The kit preferably comprises a container capable of holding the compositions separately, such as a divided bottle and a foil packet, but the compositions may be held in a single container. Preferred examples of the kit include, but are not limited to, a blister pack used for packaging tablets, capsules, or the like and a syringe or container prefilled with a drug solution.

The kit preparation may be subjected to a known sterilization treatment such as radiation and autoclaving.

### Advantageous Effects of Invention

The stomatological composition of the present invention shows at least the following effects.

1. The present invention (buccal/oral cavity administration of an NSAID) has an analgesic and anti-inflammatory effect against inflammations associated with oral cavity diseases represented by oral injuries, stomatitis, periodontal diseases, tooth pains, pains after tooth extraction, post-operative pains, gingivitis and tooth hypersensitivity and/or pains associated with the inflammations. By increasing the number of administration times, the effect may be enhanced.

2. The efficacy of ibuprofen is 6 to 8 times as mild as those of indomethacin and diclofenac, and ibuprofen is safer than indomethacin and diclofenac. Hence, in oral rinsing, which is a method of use in the present invention, ibuprofen can be used without almost no concern about the upper limit of the dose (the amount in direct contact with an affected area) and is advantageous.

3. NSAID compounds including ibuprofen may have strong irritating bitterness as they are. To overcome this problem, NSAIDs including ibuprofen used for analgesic/anti-inflammatory purposes are conventionally subjected to capsulation, coating of tablets or granules, or other treatments for the elimination of the bitterness or the like.

In contrast, as for the composition of the present invention, the strong irritating bitterness is reduced in a dissolved state of the composition. In other words, an ibuprofen liquid formulation as an embodiment of the present invention which contains about 1 to 1.3 mol of an inorganic base such as sodium hydroxide relative to 1 mol of ibuprofen used for the production of the composition and, for example, about 0.1 to 0.3 mol of an inorganic acid such as hydrochloric acid relative to 1 mol of the inorganic base is used for only holding in the oral cavity not swallowing, and thus the irritating bitterness of the active pharmaceutical ingredient is hardly found to be sensed. This is a novel bitterness reduction technique of NSAIDs in the oral cavity. The composition of the present invention can comprise a pharmaceutically acceptable carrier. In addition, by appropriately adding an additive such as water, a solvent, a gelling agent, a pH adjuster, a solubilizer, a moisturizer, a thickener, a suspending agent, a flavoring agent, a sweetening agent, a corrigent, a coloring agent, an antiseptic agent, a surfactant, an emulsifier, or a stabilizing agent, the composition can be accepted by a wide range of patients including children, and patients' physical/psychological burden can be reduced.

4. In the present invention, an NSAID including ibuprofen or a heparin compound comes in direct contact with an affected area in the oral cavity, and thus produces immediate effects. In particular, the analgesic effect on stomatitis and mild tooth pain has been confirmed as shown in examples described below.

5. When the composition of the present invention is used as a gargle (oral rinse) and an NSAID including ibuprofen or a heparin compound is not swallowed, almost no systemic side effects are thought to occur. In such a use, a patient may mistakenly swallow the whole or part of the composition, but as long as the amount of an NSAID including ibuprofen in the composition is equal to or smaller than that in commonly used oral preparations, its method of use is the same as the conventionally approved/applied method of use of NSAIDs including ibuprofen for pain relief, and therefore the composition is considered to be highly safe. The heparin compounds are all highly polar saccharides having high molecular weights and sulfuric acid groups, and thus are not absorbed through the gastrointestinal tract even when swallowed mistakenly.

6. Alternatively, only when used for patients with inflammations/pains/infections of the esophagus or the pharynx, it is acceptable that the composition of the present invention is held in the oral cavity as long as possible and then swallowed.

A most likely side effect of cancer chemotherapy or radiotherapy is thought to be stomatitis. In the clinical practice, there is no commercial, pharmaceutical product promoting effective pain relief or no appropriate therapy in the present circumstances, and current treatment is limited to an indomethacin spray or a Voltaren gargle provided as a hospital preparation in some hospitals (Non-Patent Literature 1, 2).

The present invention can be used to meet medical needs, including such a case, immediately and safely.

### DESCRIPTION OF EMBODIMENTS

### Examples

The present invention will next be described with reference to specific examples and the like, but the invention is not limited to the examples. Various changes may be made without departing from the scope of the invention.

Materials, reagents, and the like used in the present invention may be obtained as commercial products or prepared by methods known in the technical field.

### Experimental Example 1 Test 1 for Examination of Irritating Bitterness of Commercially Available Ibuprofen Preparation

Four commercially available soft capsules containing 150 mg of ibuprofen were added to 100 mL of tap water and then allowed to stand for 3 hours. During the period, the capsules disintegrated and the content liquid thereof was dispersed, resulting in a milky white suspension. The suspension was stirred, and then about 10 mL of the suspension was held in the mouth for about 30 seconds for sensory testing.

As a result, slight burning sensation (numbness and irritating bitterness), which was sufficiently tolerable for normal adults, was sensed in the mouth (n=2: n represents the number of subjects).

### Experimental Example 2 Test 2 for Examination of Irritating Bitterness of Commercially Available Ibuprofen Preparation

Commercially available soft capsules containing 150 mg of ibuprofen were cut, and the content liquid taken out of the capsules was held in the mouth for sensory testing.

As a result, irritating, stabbing pain and slight bitterness were sensed (n=1).

### Experimental Example 3 Test for Examination of Irritating Bitterness of Commercially Available Ibuprofen Preparation+Sodium Hydrogen Carbonate

To 100 mL of tap water, four commercially available soft capsules containing 150 mg of ibuprofen were added and then allowed to stand for 3 hours, giving a suspension. To the suspension, 240 mg of sodium hydrogen carbonate was added and the whole was stirred to give a clear solution. The same persons as in Experimental Example 1 held the clear solution in the mouth for sensory testing.

As a result, unlike the case with the suspension without sodium hydrogen carbonate (Experimental Example 1), no burning sensation was sensed (n=2).

### Experimental Example 4 Test 3 for Examination of Irritating Bitterness of Commercially Available Ibuprofen Preparation

The same persons as in Experimental Example 1 bit a commercially available sugar-coated tablet containing 150 mg of ibuprofen and held the tablet in the mouth for a while.

As a result, stronger burning sensation than that of the suspension in Experimental Example 1 was sensed (n=2). This result revealed that the commercially available ibuprofen tablets cannot be used as they are for the intended method of use in the present invention, that is, holding the drug in the mouth for about 30 seconds to 1 minute.

### Example 1 Preparation Example 1 of Ibuprofen Gargle (Oral Rinse) of Present Invention

To 100 mL of water, 3 g of sodium hydrogen carbonate was added and dissolved by gentle shaking and stirring. The aqueous solution was diluted 2.5-fold, and the resulting aqueous solution was diluted 5-fold (240 mg of sodium hydrogen carbonate/100 mL of water). To the aqueous solution, three commercially available soft capsules each containing 200 mg of ibuprofen, 600 mg in total, were added, and the whole was allowed to stand at room temperature for about 2 hours. During the period, the soft capsules disintegrated and released the content thereof. The whole was stirred until homogeneous, giving an ibuprofen gargle (oral rinse). As needed, insoluble substances can be filtered off, but in this case, the gargle was used without removal of insoluble substances.

TABLE 1
Formulation of Preparation Example 1 (ibuprofen content: 0.6%)

| Ingredient | Amount in 100 g of formulation |
|---|---|
| Ibuprofen (note 1) | 600 mg |
| NaHCO₃ | 240 mg |
| Purified water | q.s. |

(note 1)
As the active pharmaceutical ingredient ibuprofen, a commercial product can be used. Pure ibuprofen commercially available from, for example, Wako Pure Chemical Industries or Shiratori Pharmaceutical can be used, or ibuprofen extracted from preparations including such ibuprofen soft capsules as used in the example can also be used.

According to the package insert, the commercially available ibuprofen soft capsules contain, as additives, polysorbate 80, potassium hydroxide, gelatin, succinylated gelatin, and sugar alcohols derived from corn starch, for example.

### Example 2 Preparation Example 2 of Ibuprofen Gargle (Oral Rinse) of Present Invention

To 40 mL of purified water, 1.5 mL of 1 N sodium hydroxide was added under stirring, then 300 mg of ibuprofen (Wako Pure Chemical Industries) was further added, and the whole was stirred at room temperature so as to be homogeneous.

After about 10 minutes, undissolved ibuprofen was still observed. Further, 0.3 mL of 1 N sodium hydroxide was gradually added under stirring for complete dissolution, giving a clear aqueous solution (pH 11.5).

To the obtained aqueous solution, 0.45 mL of 0.5 N hydrochloric acid was gradually added under stirring, and purified water was added to make 50 mL, giving an ibuprofen gargle (pH 7.2).

### Example 3 Test for Examination of Irritating Bitterness of Ibuprofen Gargle (Oral Rinse) of Present Invention

In the mouth, 8 mL of the gargle prepared in Example 2 was held for about 30 seconds and then spat out, and irritation, bitterness, numbness, and the like were evaluated. Three of six persons sensed only slight irritation, which was sufficiently tolerable, and none of them involved in the test sensed bitterness or numbness (n=6).

### Example 4 Preparation Example 3 of Ibuprofen Gargle (Oral Rinse) of Present Invention

To 40 mL of purified water, 125 mg of sodium hydrogen carbonate was added under stirring and dissolved, then 300 mg of ibuprofen (Wako Pure Chemical Industries) was added, and the whole was stirred at room temperature so as to be homogeneous. After about 10 minutes, undissolved ibuprofen was still observed. Further, 832 mg of sodium hydrogen carbonate was gradually added under stirring, and additionally, 0.6 mL of 1 N sodium hydroxide was gradually added for dissolution of ibuprofen, giving a clear aqueous solution (pH 8.6). To the obtained aqueous solution, purified water was added to make 50 mL, yielding a gargle of the present invention (pH 7.9).

### Example 5 Test for Examination of Irritating Bitterness of Ibuprofen Gargle (Oral Rinse) of Present Invention

In the mouth, 8 mL of the gargle prepared in Example 4 was held for about 30 seconds and then spat out, and irritation, bitterness, numbness, and the like were evaluated. None of the persons involved in the test sensed irritation or numbness. One of six persons sensed slight bitterness, which was sufficiently tolerable, and the remaining five persons sensed no bitterness (n=6).

### Example 6 Preparation Example 4 of Ibuprofen Gargle (Oral Rinse) of Present Invention

To 40 mL of purified water, 1.5 mL of 1 N potassium hydroxide previously prepared was added under stirring, then 300 mg of ibuprofen (Wako Pure Chemical Industries) was added, and the whole was stirred at room temperature so as to be homogeneous. After about 15 minutes, undissolved ibuprofen was still observed. Further, 0.2 mL of 1 N potassium hydroxide was gradually added under stirring for complete dissolution, giving a clear aqueous solution (pH 7.6). To the obtained aqueous solution, purified water was added to make 50 mL, yielding a gargle of the present invention (pH 7.2).

### Example 7 Test for Examination of Irritating Bitterness of Ibuprofen Gargle (Oral Rinse) of Present Invention

In the mouth, 8 mL of the ibuprofen gargle prepared in Example 6 was held for about 30 seconds and then spat out, and irritation, bitterness, numbness, and the like were evaluated.

All persons involved in the test sensed slight irritation or bitterness that was a little stronger than that of the sample prepared in Example 2 but was sufficiently tolerable. None of them sensed numbness (n=6).

### Example 8 Preparation Example 5 of Ibuprofen Gargle (Oral Rinse) of Present Invention

To 750 mL of purified water, 108 mL of 1 N sodium hydroxide was added under stirring, then 18 g of ibuprofen (Shiratori Pharmaceutical, listed in Japanese Pharmacopoeia) and 1,470 mg of sodium hydrogen carbonate (Wako Pure Chemical Industries) were added, and the whole was stirred at room temperature for about 30 minutes. To the mixture, 384 g of glycerol (Sioe Pharmaceutical, listed in Japanese Pharmacopoeia) was added under stirring (preparation liquid A).

Separately, 780 mg of methyl p-hydroxybenzoate (Wako Pure Chemical Industries) and 420 mg of propyl p-hydroxybenzoate (Tokyo Chemical Industry) were ground in a mortar, then the mixture was added to an aqueous solution containing 504 g of water and 96 g of glycerol, and the whole was stirred at 35 to 40° C. for about 30 minutes, giving an antiseptic agent solution (preparation liquid B).

To the preparation liquid A, the preparation liquid B was added at room temperature under stirring, and the mixture was further stirred for 20 minutes. Next, 0.5 N hydrochloric acid was gradually added at room temperature with stirring to adjust the pH to 7.4, and then purified water was added to make 3,000 g. The resulting mixture was filtered through a disposable sterile filter (manufactured by Millipore, Sterivex (registered trademark), a pore size of 0.22 μm, similarly used in the below examples and the like), and the filtrate was placed into a sterilized brown light-resistant container. The container was sealed and stored away from light at room temperature until use.

TABLE 2
Formulation of Preparation Example 5 (ibuprofen content: 0.6%)

| Ingredient | Amount in 100 g of formulation |
|---|---|
| Ibuprofen | 600 mg |
| 1N NaOH | 144 mg/3.6 mL |
| NaHCO₃ | 49 mg |
| 0.5N HCl | q.s. |
| Glycerol | 16 g |
| Methyl p-hydroxybenzoate | 26 mg |
| Propyl p-hydroxybenzoate | 14 mg |
| Water | q.s. |

### Example 9 Test 1 for Analgesic/Anti-Inflammatory Effect (Administration to Cancer Patients with Stomatitis)

The safety of the formulation of Example 8 was confirmed by human Phase 1 study (single administration: n=9, repeated administration for 7 days (10 times administration/day): n=9), and then the formulation was administered to four patients with mild stomatitis caused by chemotherapy and/or radiotherapy against cancer. The method of use of the gargle was as follows: for one dose, 10 mL of the gargle was taken into the mouth, vigorously swished around for about 30 seconds to 1 minute, and then spat out. The maximum acceptable frequency of use was 10 times per day.

The efficacy (pain relief) of the preparation of the present invention was observed in two patients having moderately strong pain. Details are as follows:

(1) Chemotherapy and radiotherapy patient (1 case) VAS (note 2):

Before administration of investigational drug 7 cm on average (range: 6 to 8 cm)

Day 3 of administration 5 cm on average (range: 4.5 to 5.5 cm)

Time to onset of effect: 15 minutes, duration of effect: 15 minutes

(2) Chemotherapy patient (1 case) VAS (note 2):

Before administration of investigational drug 6 cm on average (range: 4 to 7 cm)

Day 3 of administration 3 cm on average (range: 2.5 to 7 cm)

Day 4 of administration 3 cm on average (range: 3 to 4.5 cm)

Day 5 of administration 1.5 cm on average (range: 1.5, 1.5 cm)

Time to onset of effect: 18 minutes, duration of effect: 30 minutes

(note 2) VAS is the abbreviation of Visual Analogue Scale described in Clinical Guidelines for Cancer Pain Management, 2010 (Japanese Society of Palliative Medicine) or the like, and is currently the most commonly used evaluation method. In the evaluation, the intensity of an existing pain is represented as a distance from one endpoint on a 10-cm straight line, where 0 cm is indicative of “no pain” and 10 cm is indicative of “the strongest pain ever experienced”.

### Example 10 Test 2 for Analgesic/Anti-Inflammatory Effect (Administration to Stomatitis Patient)

To a mild stomatitis patient, 10 mL of the formulation prepared in accordance with Example 1 was administered before bedtime (holding time in the mouth: about 30 seconds), and the analgesic effect was observed. Neither oral cavity irritation nor bitterness was sensed. The next morning, the patient reported significantly reduced stomatitis pain.

### Example 11 Test 3 for Analgesic/Anti-Inflammatory Effect (Administration to Patient with Tooth Pain)

To a patient with mild tooth pain caused by stimulation in a cracked tooth, 10 mL of the gargle (oral rinse) of the present invention that was the formulation prepared in accordance with Example 1 was administered (holding time in the mouth: about 30 seconds). The analgesic effect was observed, and the patient reported neither oral cavity pain nor irritating bitterness. Immediately after gargling (oral rinsing), the sense of stimulation was also reduced. After the first administration, the administration was repeated twice every several minutes, three times in total, and consequently the tooth pain was further reduced.

### Example 12 Test 4 for Analgesic/Anti-Inflammatory Effect (Administration to Patient with Tooth Pain)

A patient had severe pain in a decayed tooth under treatment and held the gargle (oral rinse) of Example 1 in the mouth for about 1 minute. Within 1 minute, the severe pain was relieved. The pain recurred after 2 to 3 minutes, and the gargle was held in the mouth once again for about 1 minute. As a result, the pain was relieved. After that, the pain did no recur (n=1).

### Example 13 Test 5 for Analgesic/Anti-Inflammatory Effect (Administration to Gingivitis Patient)

A patient with mild gingivitis caused by residual food between teeth held 10 mL of the gargle (oral rinse) of Example 1 in the mouth for about 1 minute. Such a treatment before bedtime reduced pain at the time of biting the next morning (n=1).

### Example 14 Preparation Example of Meloxicam Gargle (Oral Rinse) of Present Invention

To an aqueous solution of 136 mg of sodium hydrogen carbonate (Wako Pure Chemical Industries) in 5 mL of purified water, 2.9 mg of meloxicam (Tokyo Chemical Industry) was added, and the whole was shaken by hand for complete dissolution of the added meloxicam, giving a slightly pale yellow aqueous solution. To the aqueous solution, 3.3 mL of glycerol (Sioe Pharmaceutical) was added, and the whole was thoroughly shaken and stirred to yield a meloxicam gargle, of which the bitterness was reduced to a level tolerable for oral rinse. The obtained gargle had a pH of 8.5 (measured with a pH test paper, hereinafter the same applies in Examples). In the present specification, “the bitterness was reduced to a level tolerable for oral rinse” means such bitterness that an adult can hold a drug in the oral cavity for about 30 seconds to 1 minute.

### Example 15 Preparation Example of Loxoprofen Gargle (Oral Rinse) of Present Invention

In 10 mL of tap water, 40 mg of sodium hydrogen carbonate (Kenei Pharmaceutical: listed in Japanese Pharmacopoeia) was dissolved, then a loxoprofen tablet, “KUNIHIRO” (containing 60 mg of loxoprofen sodium) was added, and the whole was allowed to stand at room temperature for 1 hour. During the period, the tablet completely disintegrated. The resulting mixture was thoroughly shaken and then allowed to stand, and the supernatant was filtered. To the filtrate, 5 mL of glycerol (Sioe Pharmaceutical) was added, and the whole was thoroughly shaken and stirred, yielding a loxoprofen gargle, of which the bitterness was reduced to a level tolerable for oral rinse. The obtained gargle had a pH of 8.5.

### Example 16 Preparation Example of Celecoxib Gargle (Oral Rinse) of Present Invention

To 4 mL of 0.1 N sodium hydroxide, 12.3 mg of Selecox (Tokyo Chemical Industry) was added, and the whole was thoroughly shaken by hand and then heated on a water bath at 60° C. with occasional shaking for dissolution. The resulting aqueous solution was neutralized, under stirring, with 0.1 N hydrochloric acid to a pH of 6. In order to dissolve the precipitate formed by neutralization, 0.3 mL of Tween 20 was added to 5 mL of the suspension, and the whole was thoroughly shaken. To the resulting solution, 1.7 mL of glycerol was added, and the whole was thoroughly stirred, yielding a celecoxib gargle, of which the bitterness was reduced to a level tolerable for oral rinse. The obtained gargle had a pH of 8.0.

### Example 17 Preparation Example of Flurbiprofen Gargle (Oral Rinse) of Present Invention

To 50 mL of purified water containing 500 μL of 1N NaOH, 60.7 mg of flurbiprofen (Tokyo Chemical Industry) was added and was dissolved by stirring with occasional sonication at room temperature. Next, 0.5 N HCl was added dropwise under stirring (pH 5.7), and then sodium hydrogen carbonate was gradually added with stirring, yielding a flurbiprofen gargle, which had no irritation or bitterness and was usable for oral rinse. The obtained gargle had a pH of 8.0.

### Example 18 Preparation Example of Heparinoid Gargle (Oral Rinse) of Present Invention

To 50 mL of commercially available natural water, 1.5 g of a commercially available heparinoid-containing gel (containing 4.5 mg of heparinoid) was added. The whole was thoroughly shaken until the gel shape disappeared, and mixed and stirred until homogeneous, yielding a heparinoid containing gargle for buccal/oral cavity administration.

### Example 19 Test 6 for Analgesic/Anti-Inflammatory Effect (Administration to Gingivitis Patient)

A patient with mild gingivitis caused by residual food between teeth held 5 mL of the gargle (oral rinse) of Example 14 in the mouth for about 1 minute while occasionally swishing it around the mouth for sufficient contact with the affected area. Such a treatment before bedtime reduced pain at the time of biting the next morning (n=1).

### Example 20 Test 7 for Analgesic/Anti-Inflammatory Effect (Administration to Gingivitis Patient)

A patient with mild gingivitis caused by residual food between teeth held 10 mL of the gargle (oral rinse) of Example 15 in the mouth for about 1 minute while occasionally swishing it around the mouth for sufficient contact with the affected area. Such a treatment before bedtime reduced pain at the time of biting the next morning (n=1).

### Example 21 Test 8 for Analgesic/Anti-Inflammatory Effect (Administration to Gingivitis Patient)

A patient with mild gingivitis caused by residual food between teeth held 5 mL of the gargle (oral rinse) of Example 16 in the mouth for about 1 minute while occasionally swishing it around the mouth for sufficient contact with the affected area. After that, the same treatment was performed twice before bedtime, and pain at the time of biting was reduced the next morning (n=1).

### Example 22 Test 9 for Analgesic/Anti-Inflammatory Effect (Administration to Gingivitis Patient)

A patient with mild gingivitis caused by residual food between teeth held 10 mL of the gargle (oral rinse) of Example 17 in the mouth for about 1 minute while occasionally swishing it around the mouth for sufficient contact with the affected area. Such a treatment before bedtime reduced pain at the time of biting the next morning (n=1).

### Example 23 Test 10 for Analgesic/Anti-Inflammatory Effect (Administration to Gingivitis Patient)

A patient with mild gingivitis caused by residual food between teeth held 10 mL of the gargle (oral rinse) of Example 18 in the mouth for about 1 minute while occasionally swishing it around the mouth for sufficient contact with the affected area. After that, the same treatment was performed four times before bedtime, and pain at the time of biting was reduced the next morning (n=1).

### Example 24 Preparation Example of Ibuprofen Dentifrice of Present Invention

By using a method known in the field, such an ibuprofen dentifrice as in Table 3 can be prepared.

### [Table 3]

### Formulation of Dentifrice

| Ingredient | Amount in 100 g of formulation |
|---|---|
| Ibuprofen | 0.5 g |
| Sorbitol | 30 g |
| Precipitated silica | 20 g |
| Glycerol | 10 g |
| Sodium lauryl sulfate | 1.5 g |
| Saccharin sodium | 0.5 g |
| Titanium dioxide | 0.5 g |
| Xanthan gum | 0.5 g |
| Sodium fluoride | 0.2 g |
| Flavoring agent, coloring agent | 1 g |
| Magnesium carbonate | 0.3 g |
| Hydrochloric acid | q.s. |
| Propyl p-hydroxybenzoate | 0.03 g |
| Water | q.s. |

### Example 25 Preparation Example of Loxoprofen Patch of Present Invention

By using a method known in the field, such a loxoprofen patch as formulated in Table 4 can be prepared.

### [Table 4]

### Formulation of Patch

| Ingredient | Amount in 100 g of formulation |
|---|---|
| Loxoprofen sodium | 3 g |
| Hydroxypropyl methyl | 7 g |
| cellulose | |
| Macrogol | 0.7 g |
| Titanium oxide | 1 g |
| Sodium hydrogen carbonate | 1 g |
| Hydrochloric acid | q.s. |
| Glycerol | 1 g |
| Sodium benzoate | 0.2 g |
| Water | q.s. |

### Example 26 Preparation Example of Flurbiprofen Suspension of Present Invention

By using a method known in the field, such a flurbiprofen suspension as formulated in Table 5 can be prepared.

### [Table 5]

### Formulation of Suspension

| Ingredient | Amount in 100 g of formulation |
|---|---|
| Flurbiprofen | 0.5 g |
| Sodium hydroxide | q.s. |
| Potassium carbonate | 0.2 g |
| Hydrochloric acid | q.s. |
| Glycerol | 20 g |
| Sodium dihydrogen phosphate | 1 g |
| Water | q.s. |

### Example 27 Preparation Example of Heparinoid-Containing Semisolid Preparation of Present Invention

By using a method known in the field, such a heparinoid-containing semisolid preparation (oral cavity liniment) as formulated in Table 6 can be prepared.

### [Table 6]

### Formulation of Semisolid Preparation (Liniment)

| | Amount in 100 g | |
|---|---|---|
| Ingredient | of formulation | |
| Heparinoid | 0.3 | g |
| Polyoxyethylene hydrogenated | 1 | g |
| castor oil | | |
| Vaseline | q.s. | |
| Liquid paraffin | q.s. | |
| Cetanol | 10 | g |
| Squalane | 5 | g |
| Sodium hydroxide | q.s. | |
| Potassium hydrogen carbonate | 1 | g |
| Hydrochloric acid | q.s. | |
| Glycerol | 10 | g |
| Chlorhexidine gluconate | 0.05 | g |

| | |
|---|---|
| Water | q.s. |

### Example 28 Preparation Example of Heparin Sodium Gum of Present Invention

By using a method known in the field, such a heparin sodium-containing gum as formulated in Table 7 can be prepared.

### [Table 7]

### Formulation of Gum

| Ingredient | Amount in 100 g of formulation |
|---|---|
| Heparin sodium | 0.5 g |
| Locust bean gum | 10 g |
| Xanthan gum | 10 g |
| Sorbitol | 1 g |
| Mannitol | 3 g |
| Xylitol | 2 g |
| Sodium hydroxide | q.s. |
| Potassium hydrogen carbonate | 1 g |
| Hydrochloric acid | q.s. |
| Glycerol | 10 g |
| Chlorhexidine gluconate | 0.05 g |
| Water | q.s. |

### Example 29 Preparation Example of Ibuprofen Spray of Present Invention

The preparation liquids A and B in Example 8 were mixed, and then the mixture was filtered through a sterile filter. The filtrate was placed into a commercially available 100-mL gun spray bottle (manufactured by Komatsu KT, model: G-163a, main body: polyester, spray part: polypropylene), yielding an ibuprofen-containing spray for buccal/oral cavity administration. At the time of use, by pulling a spray nozzle, the spray of the present invention was projected in a mist form from the tip of the spray nozzle and successfully delivered exactly to an affected area in the oral cavity.

### Example 30 Preparation Example of Ibuprofen Gel of Present Invention

To 100 mL of tap water previously warmed to about 60° C., 2 g of hydroxypropyl cellulose (HPC; manufactured by Tokyo Chemical Industry, H0386, viscosity of 2% aqueous solution at 20° C.: 150 to 400 mPa·s) was added with stirring, and the mixture was further stirred while being cooled. At about 30° C., the solids were completely dissolved to give a clear HPC gel.

The preparation liquid A and the preparation liquid B in Example 8 were mixed, and then the mixture was filtered through a sterile filter. To 6 mL of the filtrate, 4 mL of the above-prepared HPC gel was added and the whole was thoroughly stirred and shaken, yielding an ibuprofen-containing gel for buccal/oral cavity administration, which had bitterness/irritation masked to some extent and was orally usable.

### Example 31 Preparation Example of Gel of Present Invention

By a production method similar to that for ibuprofen, other gels can also be produced from different active ingredients. The formulation is as shown in Table 8.

### [Table 8]

### Formulation of Gel

| Ingredient | Amount in 100 g of formulation |
|---|---|
| Active ingredient (*1) | 1 g |
| HPC gel | 1 g |
| Polyoxyethylene alkyl ether | 0.2 g |
| Xanthan gum | 1 g |
| Sodium hydroxide | q.s. |
| Sodium hydrogen carbonate | 1 g |
| Hydrochloric acid | q.s. |
| Glycerol | 20 g |
| Methyl p-hydroxybenzoate | 26 mg |
| Propyl p-hydroxybenzoate | 14 mg |
| Water | q.s. |

(*1: loxoprofen, meloxicam, or flurbiprofen)

### Example 32 Preparation Example of Ibuprofen Jelly of Present Invention

To 50 mL of tap water previously warmed to about 70° C., 5 g of a commercially available gelatin powder (Jellice (registered trademark); manufactured by Maruha Nichiro) was added and completely dissolved by thoroughly stirring (prepared gelatin solution). The preparation liquid A and the preparation liquid B in Example 8 were mixed, and then the mixture was filtered through a sterile filter. Six milliliters of the filtrate was warmed to about 40° C., and to this, 4 mL of the prepared gelatin solution was added with stirring. The whole was thoroughly mixed and stirred, and then allowed to cool to room temperature. Next, the mixture was kept in a refrigerator (at about 2 to 5° C.) for 1 hour, yielding an ibuprofen-containing jelly for buccal/oral cavity administration, which had bitterness/irritation masked to some extent and was orally usable.

### Example 33 Preparation Example of Jelly of Present Invention

By a production method similar to that for ibuprofen, other jellies can also be produced from different active ingredients. The formulation is as shown in Table 9.

### [Table 9]

### Formulation of Jelly

| Ingredient | Amount in 100 g of formulation |
|---|---|
| Active ingredient (*2) | 0.5 g |
| Prepared gelatin solution | 40 g |
| Sodium hydroxide | q.s. |
| Sodium hydrogen carbonate | 1 g |
| Hydrochloric acid | q.s. |
| Glycerol | 16 g |
| Methyl p-hydroxybenzoate | 26 mg |
| Propyl p-hydroxybenzoate | 14 mg |
| Water | q.s. |

(*2: Loxonin, meloxicam, flurbiprofen, celecoxib, or heparinoid)

### Example 34 Preparation Example of Ibuprofen Suspension of Present Invention

A saturated aqueous solution of citric acid (Japanese Pharmacopoeia) was diluted 10-fold with tap water (prepared citric acid solution). The preparation liquid A and the preparation liquid B in Example 8 were mixed, and then the mixture was filtered through a sterile filter. To 10 mL of the filtrate, 0.5 mL of the prepared citric acid solution was added dropwise with stirring, and the mixture was thoroughly shaken, yielding an ibuprofen-containing suspension for buccal/oral cavity administration, which was in a good suspension state, had bitterness reduced to some extent, and was usable for oral cavity administration. The obtained suspension had a pH of 6.5.

### INDUSTRIAL APPLICABILITY

The present invention provides a stomatological composition and a kit each comprising an NSAID such as ibuprofen, a heparin compound, or a pharmaceutically acceptable salt thereof for the prevention, alleviation, or treatment of oral inflammations and pains/infections associated with the inflammations and of inflammatory pain diseases/infection diseases of the pharynx and/or the esophagus, and further provides a method for producing the composition. The present invention can meet medical needs immediately, simply, and safely and has great industrial applicability.

1. A stomatological composition for prevention, alleviation, or treatment of an inflammation, a pain, or an infection associated with an oral disease and/or an inflammatory pain disease or an infection of an upper respiratory tract, a pharynx, and/or an esophagus, the composition comprising
an NSAID, a heparin compound, or a pharmaceutically acceptable salt thereof, and
at least one pharmaceutically acceptable carrier.

2. The composition according to claim 1, wherein the NSAID is at least one selected from the group consisting of ibuprofen, meloxicam, loxoprofen, celecoxib, and flurbiprofen, or the heparin compound is at least one selected from the group consisting of heparin, a heparinoid, and a low-molecular-weight heparin.

3. The composition according to claim 1, wherein the composition is in the form of solution, gel, gel, patch, dentifrice, jelly, spray, gum, suspension, semisolid preparation, orally disintegrating tablet, granule, chewable tablet, effervescent tablet, or troche.

4. The composition according to claim 1, wherein the oral disease is at least one selected from the group consisting of an oral injury, stomatitis, a periodontal disease, a tooth pain, a pain after tooth extraction, a pain after oral surgery, gingivitis, tooth hypersensitivity, an inflammatory disease, an inflammatory tongue pain, an infection in an oral cavity or of a pharynx or an esophagus, an inflammation and a pain associated with dry mouth, and an inflammatory pain of a pharynx and/or an esophagus.

5. The composition according to claim 1, further comprising at least one additive selected from the group consisting of water, a solvent, a gelling agent, a pH adjuster, a solubilizer, a moisturizer, a thickener, a suspending agent, a flavoring agent, a sweetening agent, a corrigent, a coloring agent, an antiseptic agent, a surfactant, an emulsifier, and a stabilizing agent.

6. The composition according to claim 1, further comprising an additional pharmacologically active ingredient.

7. The composition according to claim 1, wherein the composition is a gargle.

8. The composition according to claim 2, wherein 100 g of the composition contains 100 μg to 20 g of ibuprofen, 2.5 μg to 500 mg of meloxicam, 30 μg to 6 g of loxoprofen, 67 μg to 13.3 g of celecoxib, or 20 μg to 4 g of flurbiprofen.

9. The composition according to claim 2, wherein 100 g of the composition contains 0.2 to 40,000 units of heparin, 0.2 to 40,000 international units of the low-molecular-weight heparin, or 1.5 μg to 3 g of the heparinoid.

10. The composition according to claim 1, wherein irritation and/or bitterness is reduced.

11. A method for producing the composition according to claim 1, the method comprising
mixing an NSAID, a heparin compound, or a pharmaceutically acceptable salt thereof with at least one acceptable carrier.

12. (canceled)

13. A kit used for prevention, alleviation, or treatment of an inflammation, a pain, or an infection associated with an oral disease and/or an inflammatory pain disease or an infection of an upper respiratory tract, a pharynx, and/or an esophagus, the kit comprising
the composition according to claim 1.

14. A method of prevention, alleviation or treatment of an inflammation, a pain, or an infection associated with an oral disease and/or an inflammatory pain disease or an infection of an upper respiratory tract, a pharynx, and/or an esophagus, which comprises administering to an animal or human in need thereof a therapeutically effective amount of an NSAID, a heparin compound, or a pharmaceutically acceptable salt thereof.

15. The method according to claim 14, wherein the NSAID is at least one selected from the group consisting of ibuprofen, meloxicam, loxoprofen, celecoxib, and flurbiprofen, or the heparin compound is at least one selected from the group consisting of heparin, a heparinoid, and a low-molecular-weight heparin.

16. The method according to claim 14, wherein the oral disease is at least one selected from the group consisting of an oral injury, stomatitis, a periodontal disease, a tooth pain, a pain after tooth extraction, a pain after oral surgery, gingivitis, tooth hypersensitivity, an inflammatory disease, an inflammatory tongue pain, an infection in an oral cavity or of a pharynx or an esophagus, an inflammation and a pain associated with dry mouth, and an inflammatory pain of a pharynx and/or an esophagus.

17. The method according to claim 14, which comprises further administering a therapeutically effective amount of at least one additive selected from the group consisting of water, a solvent, a gelling agent, a pH adjuster, a solubilizer, a moisturizer, a thickener, a suspending agent, a flavoring agent, a sweetening agent, a corrigent, a coloring agent, an antiseptic agent, a surfactant, an emulsifier, and a stabilizing agent.

18. The method according to claim 14, which comprises further administering a therapeutically effective amount of an additional pharmacologically active ingredient.
